(19) United States
(12) Patent Application Publication
(10) Pub. No.: US 2014/0302150 A1
(43) Pub. Date: Oct. 9, 2014

(54) DPP-IV INHIBITOR FORMULATIONS

(71) Applicants: Umit Cifter, Istanbul (TR); Ali Turkyilmaz, Istanbul (TR); Gulay Yelken, Istanbul (TR); Mehtap Saydam, Istanbul (TR)

(72) Inventors: Umit Cifter, Istanbul (TR); Ali Turkyilmaz, Istanbul (TR); Gulay Yelken, Istanbul (TR); Mehtap Saydam, Istanbul (TR)

(21) Appl. No.: 14/342,661

(22) PCT Filed: Sep. 5, 2012
(86) PCT No.: PCT/TR2012/000137

§ 371 Date: May 15, 2014

(30) Foreign Application Priority Data
Sep. 7, 2011 (TR) 2011/08763

(51) Int. Cl. A61K 9/16; A61K 9/20; A61K 31/4985; A61K 31/40; A61K 31/403

(52) U.S. Cl. CPC A61K 9/1635; A61K 31/40; A61K 31/403; A61K 31/4985; A61K 9/2095
USPC 424/489; 514/423; 514/412; 514/249; 264/109

(57) ABSTRACT

The present invention relates to a pharmaceutical formulation, characterized by comprising a DPP-IV inhibitor and polyvinylcaprolactam-polyvinyl acetate-polyethylene glycol graft copolymer.

### FIELD OF INVENTION

The present invention relates to a formulation comprising vildagliptin or a pharmaceutically acceptable salt of vildagliptin. The present invention particularly relates to a stable formulation of vildagliptin having desired levels of dissolution rate and solubility.

### BACKGROUND OF INVENTION

Diabetes mellitus may develop depending on many factors. Following the oral administration of glucose in glucose tolerance tests, which are conducted routinely, high plasma levels and hypoglycemia are encountered. Type 2 diabetics are under macrovascular and microvascular complication risks, including particularly coronary heart disease, stroke, peripheral vascular disease, nephropathy, neuropathy, and retinopathy. For these reasons, keeping diabetes under control has vital importance.

Inhibitors of dipeptidyl peptidase 4, also DPP-4 inhibitors or gliptins, are a class of oral hypoglycemics that block DPP-4. They can be used to treat diabetes mellitus type 2. DPP-IV is a membrane-bound serine protease and is present in a soluble form in plasma. It is present in many tissues, including vascular endothelial cells and the immune system cells (CD26, the marker for activated T cells). However, specific DPP-IV inhibition does not influence the CD26 immune activation. T-cell activation and proliferation were found related to DPP-8 and DPP-9. The increase in the endogenous GLP-1 and GIP levels obtained with the development of oral selective DPP-IV inhibitors provides a more physiologic glucose-dependent antidiabetic effect on insulin and glucagon secretion.

Vildagliptin is a dipeptidyl dipeptidase-IV (DPP-IV) inhibitor developed for use in the treatment of type 2 diabetes (non-insulin dependent diabetes). Vildagliptin inhibits the degradation of the dipeptidyl dipeptidase-IV enzyme, thereby inhibiting the effects of incretin hormones, glucagon-like peptide-1 (GLP-1), and of glucose-dependent insulinotropic peptide (GIP). The chemical designation of vildagliptin is (S)-{[(3-hydroxyadamantan-1-yl)amino]acetyl}pyrrolidine-2-carbonitrile, with the chemical structure illustrated below in Formula 1.

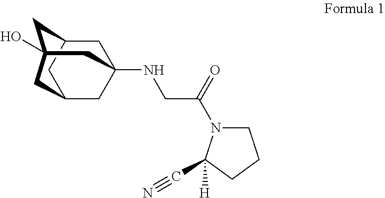

Vildagliptin is soluble in water and in organic polar solvents.

Vildagliptin is marketed under the trademark Galvus® in 50 mg dosage forms. It is used against diabetes mellitus, but particularly in treating type 2 diabetes.

There are various patents in the patent literature in relation to vildagliptin.

The patent application WO0034241 discloses vildagliptin or an acid addition salt thereof, as well as its use in diabetes mellitus and obesity.

The patent application WO2006078593 claims a direct-compression formulation of a DPP-IV inhibitor compound, and preferably of vildagliptin or an acid addition salt thereof.

The patent application W02006135723 discloses a formulation, comprising vildagliptin as an active agent, as well as hydroxypropyl methyl cellulose, microcrystalline cellulose, and magnesium stearate.

Saxagliptin is a dipeptidyl dipeptidase-IV (DPP-IV) inhibitor developed for use in the treatment of type 2 diabetes (non-insulin dependent diabetes). Saxagliptin inhibits the degradation of the dipeptidyl dipeptidase-4 enzyme, thereby inhibiting the effects of incretin hormones, glucagon-like peptide-1 (GLP-1), and of glucose-dependent insulinotropic peptide (GIP).

The chemical designation of saxagliptin is (2S,4S,5R)-2-[(2S)-2-amino-2-(3-hydroxyadamantan-1-yl)acetyl]-2-azabicyclo[3.1.0]hexane-3-carbonitrile, with the chemical structure illustrated below in Formula 2.

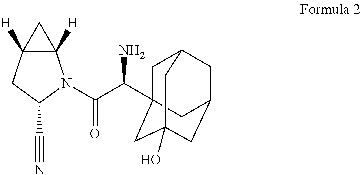 [SMILES: N#C[C@@H]1C[C@@H]2C[C@@H]2N1C(=O)[C@@H](N)C12CC3CC(CC(O)(C3)C1)C2]

Saxagliptin is marketed under the trademark ONGLYZA® in 2.5 or 5 mg dosage forms. It is used against diabetes mellitus, but particularly in treating type 2 diabetes.

There are various patents available in the patent literature in relation to saxagliptin.

The patent application U.S. Pat. No. 6,395,767 discloses saxagliptin and its use for treating purposes.

The patent application WO2010115974 describes anhydrous crystal forms of saxagliptin hydrochloride, not containing water in excess of 1.5% by weight.

The patent application WO2005115982 discloses a process for obtaining saxagliptin.

The patent application WO2005117841 discloses pharmaceutical formulations of saxagliptin, which comprise inner and outer coatings, as well as a method for obtaining that formulation.

Sitagliptin is a dipeptidyl dipeptidase-IV (DPP-IV) inhibitor developed for use in the treatment of type 2 diabetes (non-insulin dependent diabetes). Sitagliptin inhibits the degradation of the dipeptidyl dipeptidase-4 enzyme, thereby inhibiting the effects of incretin hormones, glucagon-like peptide-1 (GLP-1), and of glucose-dependent insulinotropic peptide (GIP). The chemical designation of sitagliptin is (R)-3-amino-1-[3-(trifluoromethyl)-5,6,7,8-tetrahydro[1,2,4]triazolo[4,3-a]pyrazin-7-yl]-4-(2,4,5-trifluorophenyl)butan-1-one, with the chemical structure illustrated below in Formula 3.

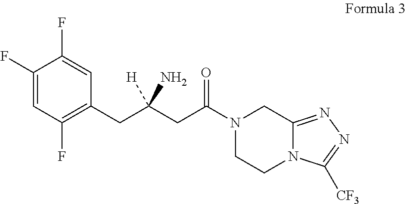 [SMILES: N[C@@H](CC(=O)N1CCn2c(nnc2C(F)(F)F)C1)Cc1cc(F)c(F)cc1F]

Sitagliptin is marketed under the trademark Januvia® in 25, 50, and 100 mg dosage forms. It is used against diabetes mellitus, but particularly in treating type 2 diabetes.

Sitagliptin is disclosed in the patent U.S. Pat. No. 6,699,871. A crystal phosphate monohydrate form of sitagliptin is disclosed in the patent WO2005003135.

The solubility and dissolution rate of vildagliptin, sitagliptin, and saxagliptin directly influence the bioavailability of these agents. For this reason, it is quite important to increase the solubility and dissolution rate of these active agents.

Another problem in relation to these active agents is stability, which emerges under the influence of ambient and physical conditions, as is the case with many other active agents. DPP-IV inhibitors are such active agents that are highly-susceptible to air and humidity. When they are exposed to air and humidity, they degrade structurally and develop chemical behavioral changes. The stability of the products developed is not at a desired level and the shelf life thereof is shortened. In addition, these active agents are reactive against the excipients employed in developing the formulations containing the same. This, in turn, causes impurities to occur in the formulations and leads to the inclusion of undesired components into the formulations.

Another problem encountered while developing formulations of said active agents is the flowability-problem, which makes the production difficult.

Based on the drawbacks and needs mentioned above, a novelty is required in the art of DPP-IV inhibitor formulations showing antidiabetic activity.

### OBJECT AND BRIEF DESCRIPTION OF INVENTION

The present invention provides an easily-administrable DPP-IV inhibitor formulation, eliminating all problems referred to above and bringing additional advantages to the relevant prior art.

Accordingly, the main object of the present invention is to obtain at least one stable formulation with antidiabetic activity.

Another object of the present invention is to provide a formulation having a desired solubility and dissolution rate, and therefore a desired level of bioavailability, with this formulation comprising a DPP-IV inhibitor produced by means of a hot-melt method.

A further object of the present invention is to eliminate the need for any liquid solvent, including water.

A further object of the present invention is to obtain a uniform formulation content.

A further object of the present invention is to develop a formulation not leading to flowability-related problems during production.

A pharmaceutical formulation is developed to carry out all objects, referred to above and to emerge from the following detailed description.

According to a preferred embodiment of the present invention, said novelty is realized with the formulation comprising DPP-IV inhibitor and polyvinylcaprolactam-polyvinyl acetate-polyethylene glycol graft copolymer.

According to another preferred embodiment of the present invention, said formulation is obtained by means of a hot-melt method not involving any liquid solvent during the granulation phase.

According to a preferred embodiment of the present invention, said DPP-IV inhibitor is at least one selected from the group comprising vildagliptin, saxagliptin, and sitagliptin.

According to a preferred embodiment of the present invention, said DPP-IV inhibitor is vildagliptin.

According to a preferred embodiment of the present invention, the proportion of vildagliptin to polyvinylcaprolactam-polyvinyl acetate-polyethylene glycol graft copolymer is in the range of 0.1 to 10, preferably 0.2 to 8, and more preferably 0.3 to 7.

Another preferred embodiment of the present invention comprises at least one or more excipients.

According to a preferred embodiment of the present invention, said excipients comprise at least one or a properly-proportioned mixture of diluents, disintegrants, glidants, lubricants, and plasticizers.

According to a preferred embodiment of the present invention, the mean particle size (d₅₀) of the granules obtained by means of the hot-melt method is in the range of 100-1000 μm, preferably 300-800 μm, and more preferably 400-600 μm.

According to a preferred embodiment, the present invention also comprises at least one or a properly-proportioned mixture of polyoxyethylene-polyoxypropylene block copolymers, stearyl macrogol glyceride, polyethylene glycol, povidone, cationic methacrylate, copovidone, methacrylic acid copolymer derivatives, cellulose acetate phthalate, acetylated monoglyceride, dibutyl tartrate, diethyl phthalate, dimethyl phthalate, glycerin, propylene glycol, triacetine, triacetine citrate and tripropionin. The polymers having a low glass transition temperature are preferred in the present invention.

In a preferred embodiment of the present invention, said disintegrant comprises at least one or a properly-proportioned mixture of polyvinylpyrrolidone and sodium starch glycolate.

In a preferred embodiment of the present invention, said glidant is colloidal silicon dioxide.

In a preferred embodiment of the present invention, said lubricant preferably comprises at least one or a properly-proportioned mixture of polyethylene glycol and magnesium stearate.

In a preferred embodiment of the present invention, said plasticizer comprises preferably at least one or a properly-proportioned mixture of castor oil, glycerin, citrate esters (acetyl tri-n-butyl citrate, acetyl triethyl citrate, tri-n-butyl citrate, triethyl citrate) dibutyl sebacate, triacetine, diethyl phthalate, low molecular weight polyethylene glycols. The use of a plasticizer serves to reduce the glass transition temperature of the polymer and to increase the stability of active agents used in the formulation.

In a preferred embodiment according to the present invention, said pharmaceutical formulation consist of,
a. vildagliptin or a pharmaceutically acceptable salt of vildagliptin at 5 to 60% by weight,
b. polyvinylcaprolactam-polyvinyl acetate-polyethylene glycol graft copolymer at 5 to 50% by weight,
c. croscarmellose sodium at 0.25 to 20% by weight,
d. colloidal silicon dioxide at 0.1 to 1% by weight,
e. magnesium stearate at 0.1 to 3% by weight,
f. plasticizer at 0.1 to 10% by weight.

In a preferred embodiment according to the present invention, said pharmaceutical formulation consist of,
a. vildagliptin or a pharmaceutically acceptable salt of vildagliptin at 5 to 60% by weight,
b. stearyl macrogol glycerides at 5 to 50% by weight,
c. croscarmellose sodium at 0.25 to 20% by weight,
d. colloidal silicon dioxide at 0.1 to 1% by weight,
e. magnesium stearate at 0.1 to 3% by weight,
f. plasticizer at 0.1 to 10% by weight.

Another preferred embodiment according to the present invention provides a method for preparing said pharmaceutical formulation, this method comprising the steps of
a. mixing vildagliptin, polyvinylcaprolactam-polyvinyl acetate-polyethylene glycol graft copolymer and plasticizer together, melting this mixture, and passing it through an extruder or sieve,
b. adding first croscarmellose sodium and colloidal silicon dioxide, and then magnesium stearate to the granules obtained and mixing the same,
c. performing a compression step on this powder mixture in a tablet machine, or filling this powder mixture into capsules.

Another preferred embodiment according to the present invention provides a method for preparing said pharmaceutical formulation, this method comprising the steps of
a. mixing vildagliptin, stearyl macrogol glycerides and plasticizers together, melting this mixture, and passing it through a sieve or an extruder,
b. adding first croscarmellose sodium and colloidal silicon dioxide, and then magnesium stearate to the granules obtained and mixing the same,
c. performing a compression step on this powder mixture in a tablet machine, or filling this powder mixture into capsules.

In another preferred embodiment of the present invention, said formulation is in the form of a tablet or capsule.

### DETAILED DESCRIPTION OF INVENTION

### Example 1

### Capsule or Tablet

| Ingredients | % amount (mg) |
|---|---|
| vildagliptin | 5-60% |
| polyvinylcaprolactam-polyvinyl | 5-50% |
| acetate-polyethylene glycol | |
| graft copolymer | |
| croscarmellose sodium | 0.25-20% |
| colloidal silicon dioxide | 0.1-1% |
| magnesium stearate | 0.1-3% |
| plasticizer | 0.1-10% |

The production of the formulation is carried out as follows: Vildagliptin, plasticizer and polyvinylcaprolactam-polyvinyl acetate-polyethylene glycol graft copolymer are mixed together, this mixture is melted and passed through an extruder or sieve. First croscarmellose sodium and colloidal silicon dioxide, and then magnesium stearate are added to the granules obtained and the resulting mixture is mixed. A compression step is performed on this powder mixture in a tablet machine, or this powder mixture is filled into capsules. The tablets are coated preferably with a humidity-barrier coating material, such as Opadry amb/Kollicoat IR.

### Example 2

| Ingredients | % amount (mg) |
|---|---|
| vildagliptin | 5-60% |
| stearyl macrogol glycerides | 5-50% |
| croscarmellose sodium | 0.25-20% |
| colloidal silicon dioxide | 0.1-1% |
| magnesium stearate | 0.1-3% |
| plasticizer | 0.1-10% |

This formulation is produced as follows: Vildagliptin, plasticizer and stearyl macrogol glycerides are mixed together, this mixture is melted and passed through an extruder or sieve. First croscarmellose sodium and colloidal silicon dioxide, and then magnesium stearate are added to the granules obtained and the resulting mixture is mixed. A compression step is performed on this powder mixture in a tablet machine, or this powder mixture is filled into capsules. The tablets are coated preferably with a humidity-barrier coating material, such as Opadry amb/Kollicoat IR.

### Example 3

### Capsule or Tablet

| Ingredients | % amount (mg) |
|---|---|
| saxagliptin | 0.2-10% |
| polyvinylcaprolactam-polyvinyl | 5-70% |
| acetate-polyethylene glycol | |
| graft copolymer | |
| croscarmellose sodium | 0.25-20% |
| colloidal silicon dioxide | 0.1-1% |
| magnesium stearate | 0.1-3% |
| plasticizer | 0.1-10% |

The production of the formulation is carried out as follows: Saxagliptin, plasticizer and polyvinylcaprolactam-polyvinyl acetate-polyethylene glycol graft copolymer are mixed together, this mixture is melted and passed through an extruder or sieve. First croscarmellose sodium and colloidal silicon dioxide, and then magnesium stearate are added to the granules obtained and the resulting mixture is mixed. A compression step is performed on this powder mixture in a tablet machine, or this powder mixture is filled into capsules. The tablets are coated preferably with a humidity-barrier coating material, such as Opadry amb/Kollicoat IR.

| Ingredients | % amount (mg) |
|---|---|
| saxagliptin | 0.2-10% |
| stearyl macrogol glycerides | 5-70% |
| croscarmellose sodium | 0.25-20% |
| colloidal silicon dioxide | 0.1-1% |
| magnesium stearate | 0.1-3% |
| plasticizer | 0.1-10% |

### Example 4

This formulation is produced as follows: Saxagliptin, plasticizer and stearyl macrogol glycerides are mixed together, this mixture is melted and passed through an extruder or sieve. First croscarmellose sodium and colloidal silicon dioxide, and then magnesium stearate are added to the granules obtained and the resulting mixture is mixed. A compression step is performed on this powder mixture in a tablet machine, or this powder mixture is filled into capsules. The tablets are coated preferably with a humidity-barrier coating material, such as Opadry amb/Kollicoat IR.

### Example 5

### Capsule or Tablet

| Ingredients | % amount (mg) |
|---|---|
| sitagliptin | 5-70% |
| polyvinylcaprolactam-polyvinyl | 5-50% |
| acetate-polyethylene glycol | |
| graft copolymer | |
| croscarmellose sodium | 0.25-20% |
| colloidal silicon dioxide | 0.1-1% |
| magnesium stearate | 0.1-3% |
| plasticizer | 0.1-10% |

The production of the formulation is carried out as follows: Sitagliptin, plasticizer and polyvinylcaprolactam-polyvinyl acetate-polyethylene glycol graft copolymer are mixed together, this mixture is melted and passed through an extruder or sieve. First croscarmellose sodium and colloidal silicon dioxide, and then magnesium stearate are added to the granules obtained and the resulting mixture is mixed. A compression step is performed on this powder mixture in a tablet machine, or this powder mixture is filled into capsules. The tablets are coated preferably with a humidity-barrier coating material, such as Opadry amb/Kollicoat IR.

| Ingredients | % amount (mg) |
|---|---|
| sitagliptin | 5-70% |
| stearyl macrogol glycerides | 5-50% |
| croscarmellose sodium | 0.25-20% |
| colloidal silicon dioxide | 0.1-1% |
| magnesium stearate | 0.1-3% |
| plasticizer | 0.1-10% |

### Example 6

This formulation is produced as follows: Sitagliptin, plasticizer and stearyl macrogol glycerides are mixed together, this mixture is melted and passed through an extruder or sieve. First croscarmellose sodium and colloidal silicon dioxide, and then magnesium stearate are added to the granules obtained and the resulting mixture is mixed. A compression step is performed on this powder mixture in a tablet machine, or this powder mixture is filled into capsules. The tablets are coated preferably with a humidity-barrier coating material, such as Opadry amb/Kollicoat IR.

With this invention, a stable formulation is surprisingly obtained which has a high solubility and dissolution rate. Said formulation comprises a DPP-IV inhibitor and polyvinylcaprolactam-polyvinyl acetate-polyethylene glycol graft copolymer. The DPP-IV inhibitor is preferably vildagliptin, saxagliptin and sitagliptin. The method described above both serves to provide a uniform formulation content, and eliminates the need for any liquid solvent including water. Any flowability-related problems are also prevented with this production method. In said formulation, the proportion of vildagliptin to polyvinylcaprolactam-polyvinyl acetate-polyethylene glycol graft copolymer is in the range of 0.1 to 10, preferably 0.2 to 8, and more preferably 0.3 to 7. These ranges allow to achieve the desired dissolution rate and solubility. Polymers with low glass transition temperature and melting temperature are used in said formulation. On the other hand, using a plasticizer which reduces the glass transition temperature increases the stability of the active agent. The plasticizer used in a hot-melt method drops down the glass transition temperature of the polymers used in hot-melting, and thus allows to formulate the active agent at lower temperatures. In result, the formulation is made more stable. The plasticizer is preferably triacetine and/or diethyl phthalate.

The pharmaceutical compositions according to the present invention may also comprise one or more pharmaceutically acceptable excipient(s). Such pharmaceutically acceptable excipients include, but are not limited to fillers, glidants, lubricants, disintegrants, surface active agents etc. and the mixtures thereof.

The present invention is used for preventing or treating diabetes mellitus in mammalians, and particularly in humans.

In this context, the term formulation may both correspond to a formulation, and to a combined meaning of the formulation and the package or blister in which the formulation is stored.

In this context, the term particle comprises a powder, granule, and a pellet.

It is also possible to use the following additional excipients in this formulation.

Lubricants, but are not restricted at least one or a mixture of sodium stearyl fumarate, polyethylene glycol, stearic acid, metal stearates, boric acid, sodium chloride benzoate and acetate, sodium or magnesium lauryl sulfate, etc.

Preservatives, but are not restricted at least one or a mixture of methyl paraben and propyl paraben and salts thereof (e.g. sodium or potassium salts), sodium benzoate, citric acid, benzoic acid, butylated hydroxytoluene and butylated hydroxyanisole.

Surface active agents, but are not restricted at least one or a mixture of sodium lauryl sulfate, dioctyl sulfosuccinate, polysorbates and polyoxyethylene alkyl esters and ethers thereof, glyceryl monolaurate saponins, sorbitan laurate, sodium lauryl sulfate, magnesium lauryl sulfate.

The present invention is hereby disclosed by referring to exemplary embodiments hereinabove. Whilst these exemplary embodiments does not restrict the object of the present invention, it must be assessed under the light of the foregoing detailed description.

1. A pharmaceutical formulation, characterized by comprising a DPP-IV inhibitor and polyvinylcaprolactam-polyvinyl acetate-polyethylene glycol graft copolymer.

2. The pharmaceutical formulation according to claim 1, wherein said formulation is obtained by means of a hot-melt method not involving any liquid solvent during the granulation phase.

3. The pharmaceutical formulation according to claim 1, wherein said DPP-IV inhibitor is at least one selected from the group comprising vildagliptin, saxagliptin, and sitagliptin.

4. The pharmaceutical formulation according to claim 3, wherein said DPP-IV inhibitor is vildagliptin.

5. The pharmaceutical formulation according to claim 4, wherein the proportion of vildagliptin to polyvinylcaprolactam-polyvinyl acetate-polyethylene glycol graft copolymer is in the range of 0.1 to 10, preferably 0.2 to 8, and more preferably 0.3 to 7.

6. The pharmaceutical formulation according to claim 1, further comprising at least one or more than one excipient.

7. The pharmaceutical formulation according to claim 6, wherein said excipient comprises at least one or a properly-proportioned mixture of diluents, binders, disintegrants, glidants, lubricants, and plasticizers.

8. The pharmaceutical formulation according to claim 2, wherein the mean particle size (d₅₀) of the granules obtained by means of the hot-melt method is in the range of 100-1000 μm, preferably 300-800 μm, and more preferably 400-600 μm.

9. The pharmaceutical formulation according to claim 1, further comprising at least one or a properly-proportioned mixture of polyoxyethylene-polyoxypropylene block copolymers, stearyl macrogol glyceride, polyethylene glycol, povidone, cationic methacrylate, copovidone, methacrylic acid copolymer derivatives, cellulose acetate phthalate, acetylated monoglyceride, dibutyl tartrate, diethyl phthalate, dimethyl phthalate, glycerin, propylene glycol, triacetine, triacetine citrate and tripropionin.

10. The pharmaceutical formulation according to claim 7, wherein said disintegrant is at least one or a properly-proportioned mixture of croscarmellose sodium and sodium starch glycolate.

11. The pharmaceutical formulation according to claim 7, wherein said glidant is colloidal silicon dioxide.

12. The pharmaceutical formulation according to claim 7, wherein said lubricant preferably comprises at least one or a properly-proportioned mixture of polyethylene glycol and magnesium stearate.

13. The pharmaceutical formulation according to claim 7, wherein said plasticizer comprises preferably at least one or a properly-proportioned mixture of castor oil, glycerin, citrate esters (acetyl tri-n-butyl citrate, acetyl triethyl citrate, tri-n-butyl citrate, triethyl citrate) dibutyl sebacate, triacetine, diethyl phthalate, low molecular weight polyethylene glycols.

14. The pharmaceutical formulation according to claim 1, consisting of,
a. vildagliptin or a pharmaceutically acceptable salt of vildagliptin at 5 to 60% by weight,
b. polyvinylcaprolactam-polyvinyl acetate-polyethylene glycol graft copolymer at 5 to 50% by weight,
c. croscarmellose sodium at 0.25 to 20% by weight,
d. colloidal silicon dioxide at 0.1 to 1% by weight,
e. magnesium stearate at 0.1 to 3% by weight, and
f. plasticizer at 0.1 to 10% by weight.

15. The pharmaceutical formulation according to claim 1, consisting of,
a. vildagliptin or a pharmaceutically acceptable salt of vildagliptin at 5 to 60% by weight,
b. stearyl macrogol glycerides at 5 to 50% by weight,
c. croscarmellose sodium at 0.25 to 20% by weight,
d. colloidal silicon dioxide at 0.1 to 1% by weight,
e. magnesium stearate at 0.1 to 3% by weight, and
f. plasticizer at 0.1 to 10% by weight.

16. A method for preparing a pharmaceutical formulation according to claim 4, comprising the steps of
a. mixing vildagliptin, plasticizer and polyvinylcaprolactam-polyvinyl acetate-polyethylene glycol graft copolymer together, melting this mixture, and passing it through an extruder or sieve,
b. adding first croscarmellose sodium and colloidal silicon dioxide, and then magnesium stearate to the granules obtained and mixing the same, and
c. performing a compression step on this powder mixture in a tablet machine, or filling this powder mixture into capsules.

17. A method for preparing a pharmaceutical formulation according to claim 4, comprising the steps of
a. mixing vildagliptin, plasticizer and stearyl macrogol glycerides together, melting this mixture, and passing it through an extruder or a sieve,
b. adding first croscarmellose sodium and colloidal silicon dioxide, and then magnesium stearate to the granules obtained and mixing the same, and
c. performing a compression step on this powder mixture in a tablet machine, or filling this powder mixture into capsules.

18. A method for preventing or treating diabetes mellitus in a mammal, particularly a human, comprising administering the pharmaceutical formulation according to claim 1 to the mammal.

19. The pharmaceutical formulation according to claim 1 in the form of a tablet or capsule.

20. The pharmaceutical formulation according to claim 2, wherein said DPP-IV inhibitor is at least one selected from the group comprising vildagliptin, saxagliptin, and sitagliptin.
